(19) United States
(12) Patent Application Publication
(10) Pub. No.: US 2021/0145778 A1
(43) Pub. Date: May 20, 2021

(54) Stable Liquid Compositions of Melphalan

(71) Applicant: Orbicular Pharmaceutical Technologies Pvt. Ltd.

(72) Inventors: Hiren Patel, Hyderabad, Telangana (IN); Bhaveshkumar Vallabhbhai Patel, Hyderabad, Telangana (IN); Raghu Kannekanti, Hyderabad, Telangana (IN); Babuji Kantu, Hyderabad, Telangana (IN); Mahesh Sanka, Hyderabad, Telangana (IN)

(73) Assignees: Orbicular Pharmaceutical Technologies Pvt. Ltd., Hyderabad, Telangana (IN); Orbicular Pharmaceutical Technologies Pvt. Ltd., Hyderabad, Telangana (IN)

(21) Appl. No.: 16/959,368

(22) PCT Filed: Dec. 27, 2018
(86) PCT No.: PCT/IB2018/060639

§ 371 Date: Jun. 30, 2020

(30) Foreign Application Priority Data
Jan. 1, 2018 (IN) 201841000059

(51) Int. Cl. A61K 31/198; A61K 47/26; A61K 47/10; A61K 47/22; A61K 47/20; A61K 9/08; A61K 9/00

(52) U.S. Cl. CPC A61K 31/198; A61K 47/26; A61K 47/10; A61K 9/0019; A61K 47/20; A61K 9/08; A61K 47/22

(57) ABSTRACT

The present invention relates to a stable non-aqueous, ready to dilute liquid pharmaceutical composition comprising (i) melphalan or a pharmaceutically acceptable salt thereof and (ii) polyoxyethylene sorbitan fatty acid esters; wherein the weight ratio of melphalan to polyoxyethylene sorbitan fatty acid esters is from about 1:0.75 to about 1:20; wherein, composition comprising melphalan, at a concentration of about 50 mg/ml to about 150 mg/ml; wherein, when the composition is diluted to produce melphalan at a concentration of about 0.1 to about 3 mg/ml, is physically and chemically stable up to about 4 hours.

### FIELD OF THE INVENTION

The present invention relates to a stable non-aqueous, ready to dilute liquid pharmaceutical composition comprising melphalan or a pharmaceutically acceptable salt thereof and polyoxyethylene sorbitan fatty acid esters, wherein the weight ratio of melphalan to polyoxyethylene sorbitan fatty acid esters is from about 1:0.75 to about 1:20.

### BACKGROUND OF THE INVENTION

Melphalan, also known as L-phenylalanine mustard, L-PAM, or L-sarcolysin is a phenylalanine derivative of nitrogen mustard. Melphalan is a class of DNA alkylating oncology agents that are administered by injection. Melphalan was initially approved as Alkeran® and is indicated for palliative treatment of patients with multiple myeloma for whom oral therapy is not appropriate at a dose of 16 mg/m². Recently, Evomela® the improved formulation of melphalan for injection was approved by agency for conditioning treatment prior to hematopoietic progenitor (stem) cell transplantation in patients with multiple myeloma at a high-dose 100 mg/m²/day and for palliative treatment of patients with multiple myeloma for whom oral therapy is not appropriate at a dose of 16 mg/m².

Melphalan exhibit poor stability in aqueous solutions due to their rapid degradation. Thus, available marketed preparations are manufactured as dry powders. Such powders are typically obtained by lyophilization.

The first approved marketed formulation of injectable Alkeran® kit consists of two components comprising of Melphalan hydrochloride and polyvinylpyrrolidone lyophilized and a diluent comprising a mixture of sodium citrate, water for injection, propylene glycol and ethanol. The Alkeran® to be infused must be diluted to not more than 0.45 mg/ml in normal saline and infused over 15 minutes.

Another commercial formulation Evomela® comprising melphalan hydrochloride and betadex sulfobutyl ether sodium. Evomela® is diluted with 0.9% sodium chloride Injection to get a final concentration of 0.45 mg/mL and infused over 30 minutes.

The lyophilized solid products are generally reconstituted with water or other suitable diluent immediately prior to administration. Time and complexity associated with lyophilization, two step dilution adds to the overall manufacturing costs for the drug.

WO2017/002030 A1 disclose a stable, ready to dilute liquid parenteral formulation of melphalan comprising solvents selected from dimethyl acetamide, polyethylene glycol, ethanol, propylene glycol and glycerine; antioxidants selected from monothioglycerol, L-cysteine and ascorbic acid.

U.S. Pat. No. 9,259,406 B2 discloses, melphalan was formulated in 5% ethanol, 5% polysorbate 80, 90% sodium chloride, 0.9% in water

WO2017/085696 A1 discloses a stable, lyophilized formulation of melphalan comprising cyclodextrin and solvents.

U.S. patent application No. 2013/0131174 A1 discloses a solid lyophilized composition of Melphalan hydrochloride having a pH between 4 and 6.

U.S. patent application No. 2014/0005148 A1 discloses a stable liquid formulation of a nitrogen mustard comprising a non-aqueous liquid, an antioxidant, an organic acid and a source of chloride ions.

WO2012/146625 A1 disclose a lyophilized pharmaceutical preparation comprising melphalan flufenamide, or a pharmaceutically acceptable salt thereof; and at least one excipient selected from the group comprising a polysorbate; a polyethylene glycol; β-cyclodextrin; α-cyclodextrin; hydroxypropyl-β-cyclodextrin; sulfobutylether-3-cyclodextrin; lactose; benzyl alcohol; disodium succinate; propylene glycol; Cremophor EL; Dimethyl sulfoxide; D-mannitol; Trehalose; Sucrose and an amino acid.

Lyophilized powders or cakes are more difficult to inspect for particulate contamination because foreign matter may be hidden by the drug itself (as compared to clear solutions which are relatively easy to inspect either visually or by means of an automated inspection process).

The reconstitution of the lyophilized product is clinically inconvenient and healthcare provider must carefully follow the reconstitution instructions in order to achieve the desired concentration of the drug. The procedures may involve calculation and careful measurement of a diluent followed by agitation of the product to achieve a homogenous solution of the product.

Many drugs like nitrogen mustards for example, melphalan require reconstitution as they exhibit poor solution stability and must be administered to the patient promptly after reconstitution. In the event of a delay between reconstitution and administration exceeding the specified time limit per the package insert, the product must be discarded.

The main limitation associated with the Alkeran® composition is low concentration of melphalan in product vial with high concentration of propylene glycol in diluent vial. In order to deliver melphalan at a dose of 100 mg/m²/day for conditioning treatment using Alkeran® formulation, a total intake of propylene glycol will be approximately 21.6 mL which is not recommended. The high concentration of propylene glycol in Alkeran® is believed to contribute to some of the side effects like unconsciousness, lactic acidosis, hyperosmolality, arrhythmias, and cardiac arrest.

The stability of the reconstituted solutions is another limitation associated with the approved formulations as, reconstituted solutions are not stable for longer durations. The reconstituted Alkeran® for injection must be administered within 60 minutes of reconstitution. The Evomela® admixture solution is stable for only 4 hours at room temperature. Further the prior arts covering Melphalan liquid compositions are silent on stability of reconstituted solutions for longer durations.

Hence, there is a strong need to develop liquid formulations comprising high concentration of melphalan (i.e. 50 to 150 mg/ml) with low concentration of propylene glycol to minimize the propylene glycol intake as compared to Alkeran® and commercially available formulations, which upon dilution produces an infusion solution, physically and chemically stable for longer durations.

The present invention provides liquid compositions comprising high concentrations of melphalan with low concentration of propylene glycol and the reconstituted solution is stable.

### SUMMARY OF THE INVENTION

The present invention provides a stable, non-aqueous, ready to dilute, liquid compositions of melphalan, which upon dilution gives melphalan solutions, physically and chemically stable for longer duration.

The object of the present invention is to prepare a stable, non-aqueous, ready to dilute, liquid compositions of melphalan, which upon dilution gives melphalan solutions, physically and chemically stable up to about 4 hours.

The object of the present invention is to prepare a stable, non-aqueous, ready to dilute, liquid compositions comprising high concentration melphalan and low concentration propylene glycol, which upon dilution gives melphalan solutions, physically and chemically stable up to about 4 hours.

The object of the present invention is to prepare a stable, non-aqueous, ready to dilute, liquid concentrates of melphalan, which upon dilution gives melphalan solutions, physically and chemically stable up to about 4 hours.

The present invention relates to a stable, non-aqueous, ready to dilute, liquid pharmaceutical composition comprising melphalan or its pharmaceutically acceptable salts thereof and polyoxyethylene sorbitan fatty acid esters.

The present invention relates to a stable, non-aqueous, ready to dilute, liquid pharmaceutical composition comprising melphalan or its pharmaceutically acceptable salts thereof and polyoxyethylene sorbitan fatty acid esters, wherein the weight ratio of melphalan to polyoxyethylene sorbitan fatty acid esters is from about 1:0.75 to about 1:20.

The present invention relates to a stable, non-aqueous, ready to dilute liquid pharmaceutical composition comprising melphalan or its pharmaceutically acceptable salts thereof and polyoxyethylene sorbitan fatty acid esters, wherein composition comprising melphalan, at a concentration of about 50 mg/ml to about 150 mg/ml.

In another aspect a stable, non-aqueous, ready to dilute, liquid pharmaceutical composition comprising melphalan or its pharmaceutically acceptable salts thereof and polyoxyethylene sorbitan fatty acid esters, further comprising propylene glycol, polyethylene glycol, ethanol and the like combinations thereof.

In another aspect a stable, non-aqueous, ready to dilute, liquid pharmaceutical composition comprising melphalan or its pharmaceutically acceptable salts thereof and polyoxyethylene sorbitan fatty acid esters, optionally further comprising an antioxidant.

In another aspect of the present invention is to provide the use of a pharmaceutical composition comprising melphalan or its pharmaceutically acceptable salts thereof and polyoxyethylene sorbitan fatty acid esters for the palliative and conditioning treatment of patients with multiple myeloma.

### DETAILED DESCRIPTION OF THE INVENTION

The present invention relates to a stable, non-aqueous, ready to dilute, liquid pharmaceutical composition comprising melphalan or its pharmaceutically acceptable salts thereof and polyoxyethylene sorbitan fatty acid esters, wherein the weight ratio of melphalan to polyoxyethylene sorbitan fatty acid esters is from about 1:0.75 to about 1:20.

The term “Melphalan” is used in broad sense to include base and its pharmaceutically acceptable derivatives thereof.

Suitable pharmaceutically acceptable derivatives include pharmaceutically acceptable salts, solvates, hydrates, anhydrates, enantiomers, esters, isomers, polymorphs, tautomers, complexes and thereof, preferably melphalan hydrochloride and not melphalan-flufenamide.

The term “ready to dilute” melphalan composition refers to compositions that contain melphalan in dissolved or solubilized form and are intended to be used as such or upon further dilution in intravenous diluents.

The term “stable” or “stability” as used herein includes both physical and chemical stability. Stability parameters include but not limited to potency, stable pH value and other physico-chemical parameters.

The term “physical stability” refers to compositions free from particles and/or that do not significantly change during storage.

The term “chemical stability” relates to a limited formation of impurities, limited decrease in potency and the like.

In one embodiment, a stable, non-aqueous, ready to dilute liquid pharmaceutical composition comprising melphalan or a pharmaceutically acceptable salt thereof and polyoxyethylene sorbitan fatty acid esters.

In another embodiment, a stable non-aqueous, ready to dilute liquid pharmaceutical composition comprising melphalan or a pharmaceutically acceptable salt thereof and polyoxyethylene sorbitan fatty acid esters, wherein the weight ratio of melphalan to polyoxyethylene sorbitan fatty acid esters is from about 1:0.5 to about 1:25;

In another embodiment, the weight ratio of melphalan to polyoxyethylene sorbitan fatty acid esters is from about 1:0.75 to about 1:20.

In another embodiment, the weight ratio of melphalan to polyoxyethylene sorbitan fatty acid esters is from about 1:1 to about 1:15.

In another embodiment, the weight ratio of melphalan to polyoxyethylene sorbitan fatty acid esters is from about 1:1 to about 1:10.

In one embodiment a stable, non-aqueous, ready to dilute liquid pharmaceutical composition comprising melphalan or a pharmaceutically acceptable salt thereof and polyoxyethylene sorbitan fatty acid esters, wherein the composition comprising melphalan, at a concentration of about 10 mg/ml to about 300 mg/ml.

In another embodiment, the composition comprising melphalan, at a concentration of about 20 mg/ml to about 250 mg/ml.

In another embodiment, the composition comprising melphalan, at a concentration of about 30 mg/ml to about 200 mg/ml.

In another embodiment, the composition comprising melphalan, at a concentration of about 40 mg/ml to about 180 mg/ml.

In yet another embodiment, the composition comprising melphalan, at a concentration of about 50 mg/ml to about 150 mg/ml.

In yet another embodiment, the composition comprising melphalan, preferably at a concentration of about 70 mg/ml to about 120 mg/ml.

In one embodiment, the compositions of the invention were diluted to obtain melphalan at a concentration of about 0.1 mg/ml, or about 0.2 mg/ml, or about 0.3 mg/ml, or about 0.45 mg/ml, or about 0.5 mg/ml, or about 0.75 mg/ml, or about 1 mg/ml, or about 2 mg/ml, or about 3 mg/ml, or about 4 mg/ml, or about 5 mg/ml, or about 6 mg/ml or about 7.5 mg/ml or about 10 mg/ml.

In one embodiment, the compositions of the invention were diluted with normal saline solution (0.9% Sodium Chloride Injection, USP).

In another embodiment, the compositions of the invention were diluted with normal saline solution (0.9% Sodium Chloride Injection, USP) to obtain melphalan at a concentration of about 0.1 mg/ml, or about 0.2 mg/ml, or about 0.3 mg/ml, or about 0.45 mg/ml, or about 0.5 mg/ml, or about 0.75 mg/ml, or about 1 mg/ml, or about 2 mg/ml, or about 3 mg/ml, or about 4 mg/ml, or about 5 mg/ml, or about 6 mg/ml or about 7.5 mg/ml or about 10 mg/ml.

In one embodiment a stable, non-aqueous, ready to dilute liquid pharmaceutical composition comprising melphalan or a pharmaceutically acceptable salt thereof and polyoxyethylene sorbitan fatty acid esters, wherein, when the composition is diluted to obtain melphalan at a concentration of about 0.1 to about 10 mg/ml, is physically and chemically stable up to about 24 hours.

In another preferred embodiment, the composition is diluted to obtain melphalan at a concentration of about 0.1 to about 3 mg/ml, is physically and chemically stable up to about 12 hours.

In another preferred embodiment, the composition is diluted to obtain melphalan at a concentration of about 0.1 to about 3 mg/ml, is physically and chemically stable up to about 6 hours.

In another preferred embodiment, the composition is diluted to obtain melphalan at a concentration of about 0.1 to about 3 mg/ml, is physically and chemically stable up to about 4 hours.

In another preferred embodiment, the composition is diluted to obtain melphalan at a concentration of about 0.1 to about 1 mg/ml, is physically and chemically stable up to about 12 hours.

In another preferred embodiment, the composition is diluted to obtain melphalan at a concentration of about 0.1 to about 1 mg/ml, is physically and chemically stable up to about 6 hours.

In another preferred embodiment, the composition is diluted to obtain melphalan at a concentration of about 0.1 to about 1 mg/ml, is physically and chemically stable up to about 4 hours.

In another embodiment, the composition comprising the polyoxyethylene sorbitan fatty acid esters selected from the group comprising of polysorbate 20, polysorbate 40, polysorbate 60, polysorbate 80 and preferably polysorbate 80.

In one embodiment, a stable non-aqueous, ready to dilute liquid pharmaceutical composition comprising melphalan or a pharmaceutically acceptable salt thereof and polysorbate 80, wherein the weight ratio of melphalan to polysorbate 80 is from about 1:0.5 to about 1:25;

In another embodiment, the weight ratio of melphalan to polysorbate 80 is from about 1:0.75 to about 1:20.

In another embodiment, the weight ratio of melphalan to polysorbate 80 is from about 1:1 to about 1:15.

In another embodiment, the weight ratio of melphalan to polysorbate 80 is from about 1:1 to about 1:10.

In one embodiment a stable, non-aqueous, ready to dilute liquid pharmaceutical composition comprising melphalan or a pharmaceutically acceptable salt thereof and polysorbate 80, wherein the composition comprising melphalan, at a concentration of about 10 mg/ml to about 300 mg/ml.

In another embodiment, the composition comprising melphalan, at a concentration of about 20 mg/ml to about 250 mg/ml.

In another embodiment, the composition comprising melphalan, at a concentration of about 30 mg/ml to about 200 mg/ml.

In another embodiment, the composition comprising melphalan, at a concentration of about 40 mg/ml to about 180 mg/ml.

In yet another embodiment, the composition comprising melphalan, at a concentration of about 50 mg/ml to about 150 mg/ml.

In yet another embodiment, the composition comprising melphalan, preferably at a concentration of about 70 mg/ml to about 120 mg/ml.

In another embodiment, the compositions of the invention were diluted with normal saline solution (0.9% Sodium Chloride Injection, USP) to obtain melphalan at a concentration of about 0.1 mg/ml, or about 0.2 mg/ml, or about 0.3 mg/ml, or about 0.45 mg/ml, or about 0.5 mg/ml, or about 0.75 mg/ml, or about 1 mg/ml, or about 2 mg/ml, or about 3 mg/ml, or about 4 mg/ml, or about 5 mg/ml, or about 6 mg/ml or about 7.5 mg/ml or about 10 mg/ml.

In one embodiment a stable, non-aqueous, ready to dilute liquid pharmaceutical composition comprising melphalan or a pharmaceutically acceptable salt thereof and polysorbate 80, wherein, when the composition is diluted to obtain melphalan at a concentration of about 0.1 to about 10 mg/ml, is physically and chemically stable up to about 24 hours.

In another preferred embodiment, the composition is diluted to obtain melphalan at a concentration of about 0.1 to about 3 mg/ml, is physically and chemically stable up to about 12 hours.

In another preferred embodiment, the composition is diluted to obtain melphalan at a concentration of about 0.1 to about 3 mg/ml, is physically and chemically stable up to about 6 hours.

In another preferred embodiment, the composition is diluted to obtain melphalan at a concentration of about 0.1 to about 3 mg/ml, is physically and chemically stable up to about 4 hours.

In another preferred embodiment, the composition is diluted to obtain melphalan at a concentration of about 0.1 to about 1 mg/ml, is physically and chemically stable up to about 12 hours.

In another preferred embodiment, the composition is diluted to obtain melphalan at a concentration of about 0.1 to about 1 mg/ml, is physically and chemically stable up to about 6 hours.

In another preferred embodiment, the composition is diluted to obtain melphalan at a concentration of about 0.1 to about 1 mg/ml, is physically and chemically stable up to about 4 hours.

In one embodiment, a stable non-aqueous, ready to dilute liquid pharmaceutical composition comprising melphalan or a pharmaceutically acceptable salt thereof and polysorbate 20, wherein the weight ratio of melphalan to polysorbate 20 is from about 1:0.5 to about 1:25.

In another embodiment a stable, non-aqueous, ready to dilute liquid pharmaceutical composition comprising melphalan or a pharmaceutically acceptable salt thereof and polysorbate 20, wherein, when the composition is diluted to obtain melphalan at a concentration of about 0.1 to about 10 mg/ml, is physically and chemically stable up to about 24 hours.

In one embodiment, a stable non-aqueous, ready to dilute liquid pharmaceutical composition comprising melphalan or a pharmaceutically acceptable salt thereof and polysorbate 40, wherein the weight ratio of melphalan to polysorbate 40 is from about 1:0.5 to about 1:25.

In another embodiment a stable, non-aqueous, ready to dilute liquid pharmaceutical composition comprising melphalan or a pharmaceutically acceptable salt thereof and polysorbate 40, wherein, when the composition is diluted to obtain melphalan at a concentration of about 0.1 to about 10 mg/ml, is physically and chemically stable up to about 24 hours.

In one embodiment, a stable non-aqueous, ready to dilute liquid pharmaceutical composition comprising melphalan or a pharmaceutically acceptable salt thereof and polysorbate 60, wherein the weight ratio of melphalan to polysorbate 60 is from about 1:0.5 to about 1:25.

In another embodiment a stable, non-aqueous, ready to dilute liquid pharmaceutical composition comprising melphalan or a pharmaceutically acceptable salt thereof and polysorbate 60, wherein, when the composition is diluted to obtain melphalan at a concentration of about 0.1 to about 10 mg/ml, is physically and chemically stable up to about 24 hours.

In another embodiment the stable, non-aqueous, ready to dilute liquid pharmaceutical composition of the present invention may optionally comprise castor oil or derivatives such as hydrogenated castor oil and the like or mixtures thereof.

Inventors of the present invention found that the presence of polyoxyethylene sorbitan fatty acid esters at specified stoichiometric ratio in the compositions shall improve the solubility, stability of melphalan in aqueous diluent to give safe compositions at all dilution concentrations.

In one embodiment, a stable non-aqueous, ready to dilute liquid pharmaceutical composition comprising melphalan or a pharmaceutically acceptable salt thereof, polyoxyethylene sorbitan fatty acid esters and one or more solvents individually or in combination with one another.

In another embodiment solvent include but are not limited to dimethylacetamide (DMA), dimethyl sulfoxide (DMSO), N-methylpyrrolidone, dimethyl isosorbide, ethanol; glycol, such as polyethylene glycol (PEG), propylene glycol, polypropylene glycol (PPG) and the like or mixtures thereof; diol such as a straight chain, branched, or cyclic aliphatic diol; triol, such as a straight chain, branched, or cyclic aliphatic triol; polyoxyethylene ether, polyethylene glycol ether and the like or mixtures thereof.

In another embodiment, the PEG has a molecular weight ranging from about 100 g/mol to about 2,500 g/mol.

In another embodiment, the PEG has a molecular weight ranging from between about 200 g/mol to about 1000 g/mol.

In a preferred embodiment, the PEG has an average molecular weight ranging from between about 300 g/mol to about 800 g/mol.

In a preferred embodiment, exemplary PEG's include PEG-300, PEG-400, PEG-600 and PEG-800.

In another embodiment, the PEG has an average molecular weight ranging from between about 250 g/mol to about 700 g/mol.

In another embodiment, polypropylene glycol (PPG) may have a molecular weight ranging from about 200 g/mol to about 2500 g/mol.

In a preferred embodiment, exemplary poly propylene glycols include PPG-250, PPG-425, and PPG-700.

In one embodiment, diol such as a straight chain, branched, or cyclic aliphatic diol may have 2-20 carbon atoms.

In another embodiment, diols include propylene glycol (1,2-propane diol), 1,3-propane diol, 1,4-butane diol, 1,5-pentane diol, 1,2-cyclopentane diol, 1,2-cyclohexane diol and its higher aliphatic homologs, their positional isomers, derivatives or mixtures thereof.

In one embodiment, a triol such as a straight chain, branched, or cyclic aliphatic triol may have 2-20 carbon atoms.

In another embodiment, the triol solvents include glycerin (glycerol), butane 1,2,3-triol, 1,3,5-pentane triol, cyclohexane triol and cycloheptanetriol its higher aliphatic homologs, their positional isomers, derivatives or mixtures thereof.

In one embodiment, a stable, non-aqueous, ready to dilute liquid pharmaceutical composition comprising melphalan or a pharmaceutically acceptable salt thereof, polyoxyethylene sorbitan fatty acid esters and solvents include propylene glycol, polyethylene glycol, optionally ethanol and the like or combinations thereof.

In one embodiment, a stable, non-aqueous, ready to dilute liquid pharmaceutical composition comprising melphalan or a pharmaceutically acceptable salt thereof, polyoxyethylene sorbitan fatty acid esters and solvents include propylene glycol, polyethylene glycol, ethanol and the like or combinations thereof.

In one embodiment, the composition comprising propylene glycol in a range from about 5% to about 90% by weight of the composition.

In another embodiment, the composition comprising propylene glycol in a range from about 10% to about 90% by weight of the composition.

In another embodiment, the composition comprising propylene glycol in a range from about 30% to about 90% by weight of the composition.

In one embodiment, the composition comprising polyethylene glycol in a range from about 5% to about 90% by weight of the composition.

In another embodiment, the composition comprising polyethylene glycol in a range from about 10% to about 90% by weight of the composition.

In another embodiment, the composition comprising polyethylene glycol in a range from about 30% to about 90% by weight of the composition.

In another embodiment, the composition comprising ethanol in a range from about 5% to about 50% by weight of the composition.

In another embodiment, the composition comprising dimethylacetamide in a range from about 30% to about 90% by weight of the composition.

In another embodiment, the composition comprising dimethyl sulfoxide in a range from about 30% to about 90% by weight of the composition.

The stable, non-aqueous, ready to dilute, liquid pharmaceutical composition of the present invention, optionally further comprising an antioxidant.

In one embodiment, one or more antioxidants selected from the group, but not limited to butylated hydroxyanisole (BHA), butylated hydroxyl toluene (BHT), citric acid, lactic acid, benzoic acid, tocopherol (Vitamin E) and its derivatives or tocopheryl (Vitamin E) and its derivatives, ethylenediaminetetraacetic acid, sodium metabisulfite, sodium bisulfite, monothioglycerol, ascorbic acid and their esters, L-cysteine, parabens, benzyl alcohol, propyl gallate, thioglycolic acid, tartaric acid, phosphoric acid, gluconic acid, thiodipropionic acid, acetonic dicarboxylic acid, amino acids such as histidine, cysteine, tryptophan, tyrosine; chelating agents and the like or combinations thereof.

The compositions of the present invention may additionally contain buffers, pH adjusting agents, stabilizers such as, but not limited to citrate buffer, glutamate, dicarboxylic acid, lecithin, di(hydroxyethyl)glycine, sorbitol, bicarbonate, tartrate, benzoate, lactate, gluconate, glycine, TRIS buffer, acetate buffer, boric acid buffer, phosphate buffer, amino acids, meglumine and the like or combinations thereof.

The pH of the composition plays a crucial role in the stability of the nitrogen mustard formulation, as the protonation of the nitrogen in the mustard moiety avoids the formation of an aziridine ring, which is highly unstable and can result in unacceptable levels of degradation of the nitrogen mustard. An acidic pH is required to maintain the protonated state of the nitrogen in the mustard moiety.

In one embodiment, the pH of the present composition is in a range between about pH 0.5 to about pH 5.

In another embodiment, the pH is in a range between about pH 1.5 to about pH 4.5. For example, the pH is in the range between about pH 2.0 to about pH 4.0.

In one embodiment, the compositions of the present invention are stable under ambient or refrigerated storage conditions, for example from about 5° C. to about 25° C.

In one embodiment, the reconstituted compositions of the present invention are stable under ambient or refrigerated storage conditions, for example from about 5° C. to about 25° C.

In one embodiment, the composition of the present invention is substantially free of any organic acids.

In one embodiment the stable non-aqueous, ready to dilute liquid pharmaceutical composition comprising

(i) melphalan or a pharmaceutically acceptable salt thereof and

(ii) polyoxyethylene sorbitan fatty acid esters;

wherein the weight ratio of melphalan to polyoxyethylene sorbitan fatty acid esters is from about 1:0.75 to about 1:20;

wherein, composition comprising melphalan, at a concentration of about 50 mg/ml to about 150 mg/ml;

wherein, when the composition is diluted to obtain melphalan at a concentration of about 0.1 to about 3 mg/ml, is physically and chemically stable up to about 12 hours.

In another embodiment, the compositions of the invention diluted to obtain melphalan at a concentration of about 0.1 to 3 mg/ml, is physically and chemically stable up to about 6 hours.

In another embodiment, the compositions of the invention diluted to obtain melphalan at a concentration of about 0.1 to 3 mg/ml, is physically and chemically stable up to about 4 hours.

In one embodiment the stable non-aqueous, ready to dilute liquid pharmaceutical composition comprising

(i) melphalan or a pharmaceutically acceptable salt thereof and

(ii) polysorbate 80;

wherein the weight ratio of melphalan to polysorbate 80 is from about 1:0.75 to about 1:20;

wherein, composition comprising melphalan, at a concentration of about 50 mg/ml to about 150 mg/ml;

wherein, when the composition is diluted to obtain melphalan at a concentration of about 0.1 to about 1 mg/ml, is physically and chemically stable up to about 12 hours.

In another embodiment, the compositions of the invention diluted to obtain melphalan at a concentration of about 0.1 to 1 mg/ml, is physically and chemically stable up to about 6 hours.

In another embodiment, the compositions of the invention diluted to obtain melphalan at a concentration of about 0.1 to 1 mg/ml, is physically and chemically stable up to about 4 hours.

In one embodiment the stable non-aqueous, ready to dilute liquid pharmaceutical composition comprising

(i) melphalan or a pharmaceutically acceptable salt thereof and

(ii) polysorbate 80;

wherein the weight ratio of melphalan to polysorbate 80 is from about 1:0.75 to about 1:20;

wherein, composition comprising melphalan, at a concentration of about 50 mg/ml to about 150 mg/ml;

wherein, when the composition is diluted to obtain melphalan at a concentration of about 0.1 to about 3 mg/ml, is physically and chemically stable up to about 12 hours.

In another embodiment, the compositions of the invention diluted to obtain melphalan at a concentration of about 0.1 to 3 mg/ml, is physically and chemically stable up to about 6 hours.

In another embodiment, the compositions of the invention diluted to obtain melphalan at a concentration of about 0.1 to 3 mg/ml, is physically and chemically stable up to about 4 hours.

In one embodiment the stable non-aqueous, ready to dilute liquid pharmaceutical composition comprising,

(i) melphalan or a pharmaceutically acceptable salt thereof and

(ii) polysorbate 80;

wherein the weight ratio of melphalan to polysorbate 80 is from about 1:1 to about 1:10;

wherein, composition comprising melphalan, at a concentration of about 70 mg/ml to about 120 mg/ml;

wherein, when the composition is diluted to obtain melphalan, at a concentration of about 0.1 to about 3 mg/ml, is physically and chemically stable up to about 12 hours.

In another embodiment, the compositions of the invention diluted to obtain melphalan at a concentration of about 0.1 to 3 mg/ml, is physically and chemically stable up to about 6 hours.

In another embodiment, the compositions of the invention diluted to obtain melphalan at a concentration of about 0.1 to 3 mg/ml, is physically and chemically stable up to about 4 hours.

In one embodiment the stable, non-aqueous, ready to dilute, liquid pharmaceutical composition comprising,

(i) melphalan or a pharmaceutically acceptable salt thereof and

(ii) polysorbate 80;

(iii) propylene glycol;

(iv) optionally ethanol;

(v) optionally monothioglycerol;

wherein the weight ratio of melphalan to polysorbate 80 is from about 1:1 to about 1:10;

wherein, composition comprising, melphalan, at a concentration of about 70 mg/ml to about 120 mg/ml;

wherein, when the composition is diluted to obtain melphalan at a concentration of about 0.1 to about 3 mg/ml, is physically and chemically stable up to about 12 hours.

In another embodiment, the compositions of the invention diluted to obtain melphalan at a concentration of about 0.1 to 3 mg/ml, is physically and chemically stable up to about 6 hours.

In another embodiment, the compositions of the invention diluted to obtain melphalan at a concentration of about 0.1 to 3 mg/ml, is physically and chemically stable up to about 4 hours.

In one embodiment the stable, non-aqueous, ready to dilute, liquid pharmaceutical composition,

(i) melphalan or a pharmaceutically acceptable salt thereof and

(ii) polysorbate 80;

(iii) propylene glycol;

(iv) optionally ethanol;

(v) optionally tocopherol and its derivatives;

wherein the weight ratio of melphalan to polysorbate 80 is from about 1:1 to about 1:10;

wherein, composition comprising melphalan, at a concentration of about 70 mg/ml to about 120 mg/ml;

wherein, when the composition is diluted to obtain melphalan at a concentration of about 0.1 to about 3 mg/ml, is physically and chemically stable up to about 12 hours.

In another embodiment, the compositions of the invention diluted to obtain melphalan at a concentration of about 0.1 to 3 mg/ml, is physically and chemically stable up to about 6 hours.

In another embodiment, the compositions of the invention diluted to obtain melphalan at a concentration of about 0.1 to 3 mg/ml, is physically and chemically stable up to about 4 hours.

In one embodiment the stable, non-aqueous, ready to dilute, liquid pharmaceutical composition comprising,

(i) melphalan or a pharmaceutically acceptable salt thereof and

(ii) polysorbate 80;

(iii) polyethylene glycol;

(iv) optionally ethanol;

(v) optionally monothioglycerol;

wherein the weight ratio of melphalan to polysorbate 80 is from about 1:1 to about 1:10;

wherein, composition comprising melphalan, at a concentration of about 70 mg/ml to about 120 mg/ml;

wherein, when the composition is diluted to obtain melphalan at a concentration of about 0.1 to about 3 mg/ml, is physically and chemically stable up to about 12 hours.

In another embodiment, the compositions of the invention diluted to obtain melphalan at a concentration of about 0.1 to 3 mg/ml, is physically and chemically stable up to about 6 hours.

In another embodiment, the compositions of the invention diluted to obtain melphalan at a concentration of about 0.1 to 3 mg/ml, is physically and chemically stable up to about 4 hours.

In one embodiment the stable, non-aqueous, ready to dilute, liquid pharmaceutical composition comprising,

(i) melphalan or a pharmaceutically acceptable salt thereof and

(ii) polysorbate 80;

(iii) polyethylene glycol;

(iv) optionally ethanol;

(v) optionally tocopherol and its derivatives;

wherein the weight ratio of melphalan to polysorbate 80 is from about 1:1 to about 1:10;

wherein, composition comprising melphalan, at a concentration of about 70 mg/ml to about 120 mg/ml;

wherein, when the composition is diluted to obtain melphalan at a concentration of about 0.1 to about 3 mg/ml, is physically and chemically stable up to about 12 hours.

In another embodiment, the compositions of the invention diluted to obtain melphalan at a concentration of about 0.1 to 3 mg/ml, is physically and chemically stable up to about 6 hours.

In another embodiment, the compositions of the invention diluted to obtain melphalan at a concentration of about 0.1 to 3 mg/ml, is physically and chemically stable up to about 4 hours.

In one embodiment the stable non-aqueous, ready to dilute liquid pharmaceutical composition comprising

(i) melphalan or a pharmaceutically acceptable salt thereof and

(ii) polysorbate 80;

(iii) polyethylene glycol;

(iv) propylene glycol;

(iv) optionally ethanol;

(v) optionally tocopherol and its derivatives;

wherein the weight ratio of melphalan to polysorbate 80 is from about 1:1 to about 1:10;

wherein, composition comprising melphalan, at a concentration of about 70 mg/ml to about 120 mg/ml;

wherein, when the composition is diluted to obtain melphalan at a concentration of about 0.1 to about 3 mg/ml, is physically and chemically stable up to about 12 hours.

In another embodiment, the compositions of the invention diluted to obtain melphalan at a concentration of about 0.1 to 3 mg/ml, is physically and chemically stable up to about 6 hours.

In another embodiment, the compositions of the invention diluted to obtain melphalan at a concentration of about 0.1 to 3 mg/ml, is physically and chemically stable up to about 4 hours.

In one embodiment the stable non-aqueous, ready to dilute liquid pharmaceutical composition comprising

(i) melphalan or a pharmaceutically acceptable salt thereof and

(ii) polysorbate 80;

(iii) polyethylene glycol;

(iv) propylene glycol;

(iv) optionally ethanol;

(v) optionally monothioglycerol;

wherein the weight ratio of melphalan to polysorbate 80 is from about 1:1 to about 1:10;

wherein, composition comprising melphalan, at a concentration of about 70 mg/ml to about 120 mg/ml;

wherein, when the composition is diluted to obtain melphalan at a concentration of about 0.1 to about 3 mg/ml, is physically and chemically stable up to about 12 hours.

In another embodiment, the compositions of the invention diluted to obtain melphalan at a concentration of about 0.1 to 3 mg/ml, is physically and chemically stable up to about 6 hours.

In another embodiment, the compositions of the invention diluted to obtain melphalan at a concentration of about 0.1 to 3 mg/ml, is physically and chemically stable up to about 4 hours.

To further illustrate the invention, the following examples are provided. It is to be understood that these examples are provided for illustrative purposes and are not to be construed as limiting the scope of the present invention in any manner whatsoever.

### Example 1

Formulations of melphalan were initially prepared to evaluate for their ability to solubilize melphalan and stability.

TABLE 1
Non-aqueous compositions

Quantity (mg/ml)

| S. No | Ingredients | F1 | F2 | F3 | F4 |
|---|---|---|---|---|---|
| 1 | Melphalan | 100 | 90 | 100 | 100 |
| 2 | Polysorbate 80 | 150 | 300 | 200 | 300 |
| 3 | DL-A-Tocopheryl | 0.7 | 0.7 | 0.7 | 0.5 |
| | acetate | | | | |
| 4 | Propylene glycol | — | 400 | 200 | 200 |
| 5 | Ethanol | — | Qs to | — | — |
| | | | 1 mL | | |
| 6 | PEG 300 | Qs to | — | Qs to | Qs to |
| | | 1 mL | | 1 mL | 1 mL |

TABLE 2
Physical Observations

| S. No | Formulation | Description | Remarks |
|---|---|---|---|
| 1 | F1 | H | Clear initially and turned hazy |
| 2 | F2 | H | Slight hazy |
| 3 | F3 | CS | Solution was Clear |
| 4 | F4 | CS | Solution was Clear |

CS: Clear solution,
H: Hazy solution

### Example 2

Formulations of melphalan were prepared with monothioglycerol and DL-A-Tocopheryl acetate, to evaluate the physical and chemical stability of compositions.

TABLE 3
Non-aqueous compositions

Quantity (mg/ml)

| S. No | Ingredients | F5 | F6 |
|---|---|---|---|
| 1 | Melphalan | 100 | 100 |
| 2 | Polysorbate 80 | 100 | 100 |
| 3 | Propylene glycol | 200 | 200 |
| 4 | DL-A-Tocopheryl | — | 0.7 |
| | acetate | | |
| 5 | Monothioglycerol | 5 | — |
| 6 | Sodium hydroxide | 0.5 | 0.5 |
| 7 | Ethanol | — | — |
| 8 | PEG 300 | Qs to 1 mL | Qs to 1 mL |

TABLE 4
Stability data for the products obtained from Example 2

Condition
Total Impurity

| | |
|---|---|
| 2-8° C. | 25° C./60% RH |

| Time Point | Initial | 1 W | 1 M | 1 M |
|---|---|---|---|---|
| Formulation F5 | NA | 0.3 | 0.69 | 3.62 |
| Formulation F6 | 0.25 | NA | 0.29 | 1.30 |

Description

| | | | | |
|---|---|---|---|---|
| Formulation F5 | Clear | Clear | Clear | Clear |
| Formulation F6 | Clear | Clear | Clear | Clear |

From the above observations, it was concluded that higher level of impurities was observed in compositions with monothioglycerol compared to DL-A-Tocopheryl acetate. The presence of DL-A-Tocopheryl acetate in compositions significantly improved the stability of solubilized melphalan.

Further, the above data confirms that the stability of the present invention as a ready to dilute compositions, wherein the above compositions maintains physical and chemical stability to allow storage at a commercially relevant temperature, such as 2-8° C. and 25° C.

### Example 3

To determine the role of pH adjusting agent on physical and chemical stability of melphalan, compositions were prepared with and without NaOH.

TABLE 5
Non-aqueous compositions

Quantity (mg/ml)

| S. No | Ingredients | F7 | F8 |
|---|---|---|---|
| 1 | Melphalan | 100 | 100 |
| 2 | Polysorbate 80 | 100 | 100 |
| 3 | Propylene glycol | 200 | 200 |
| 4 | DL-A-Tocopheryl acetate | 0.7 | 0.7 |
| 5 | Sodium hydroxide | 0.5 | — |
| 6 | PEG 300 | Qs to 1 mL | Qs to 1 mL |

TABLE 6
Stability data for the product obtained from Example 3

| | Condition | | | | | | |
|---|---|---|---|---|---|---|---|
| | Total Impurity | | | | | | |
| | | 25° C./60% RH | | | 2-8° C. | | |
| Time Point | Initial | 1 M | 2 M | 3 M | 1 M | 2 M | 3 M |
| Formulation F7 | 0.25 | 1.3 | 2.57 | 3.53 | 0.29 | 0.44 | 0.54 |
| Formulation F8 | 0.2 | 1.13 | 2.26 | 3.16 | 0.24 | 0.37 | 0.45 |
| | Description | | | | | | |
| Formulation F7 | Clear | Clear | Clear | Clear | Clear | Clear | Clear |
| Formulation F8 | Clear | Clear | Clear | Clear | Clear | Clear | Clear |

From the above observations it was conclude that the total impurity levels were same in the compositions with and without NaOH.

Further, the above data confirms that the stability of the present invention as a ready to dilute compositions, wherein the above compositions maintains physical and chemical stability to allow storage at a commercially relevant temperature, such as 2-8° C. and 25° C.

### Example 4

Formulations of melphalan with different quantities of polysorbate 80 were prepared to evaluate physical stability.

TABLE 7
Non-aqueous compositions

| | | Quantity (mg/ml) | | |
|---|---|---|---|---|
| S. No | Ingredients | F9 | F10 | F11 |
| 1 | Melphalan | 100 | 100 | 100 |
| 2 | Polysorbate 80 | 100 | 200 | 300 |
| 3 | DL-A-Tocopheryl | — | 0.7 | 0.5 |
| | acetate | | | |
| 4 | Monothioglycerol | 5 | — | — |
| 5 | Propylene glycol | 200 | 200 | 200 |
| 6 | Sodium hydroxide | 0.5 | 0.5 | 0.5 |
| 7 | PEG 300 | Qs to 1 mL | Qs to 1 mL | Qs to 1 mL |

TABLE 8
Physical Observations after dilution with 0.9% Sodium chloride

| | | Dilution with | | | |
|---|---|---|---|---|---|
| | Formu- | 0.9% Sodium | | | |
| S. No | lation | Chloride solution | | Description | Remarks |
| 1 | F9 | 0.45 | mg/ml | CS | Solution was clear |
| | | 1 | mg/ml | H | Solution was not |
| | | 2 | mg/ml | H | clear |
| 2 | F10 | 0.45 | mg/ml | CS | Solution was clear |
| | | 1 | mg/ml | CS | |
| | | 2 | mg/ml | H | Solution was not |
| | | | | | clear |
| 3 | F11 | 0.45 | mg/ml | CS | Solution was Clear |
| | | 1 | mg/ml | CS | |
| | | 2 | mg/ml | CS | |

CS: Clear solution,
H: Hazy solution

From the above observations it was concluded that, all the compositions comprising different quantities of polysorbate 80 were clear. The above formulations were further diluted with 0.9% sodium chloride solution to obtain physiological solutions comprising melphalan at 0.45, 1 and 2 mg/ml solutions.

All the diluted compositions at 0.45 mg/ml melphalan were clear. The diluted compositions of F9 with higher concentration of melphalan (for ex. 1 and 2 mg/ml) was not clear after dilution.

The diluted compositions of F10 with higher concentration of melphalan (for ex. 1 mg/ml) was clear. Another concentration, 2 mg/mL after dilution was not clear.

The dilution compositions of F11 with high concentrations of melphalan, for example 1 and 2 mg/ml were physically stable i.e. clear and free from any drug particles, hence it was concluded that higher concentration of polysorbate 80 was necessary to obtain stable compositions of melphalan.

1-10. (canceled)

11. A concentrated, non-aqueous pharmaceutical composition comprising:
melphalan or a pharmaceutically acceptable salt thereof; and
a polyoxyethylene sorbitan fatty acid ester;
wherein the ratio of melphalan to polyoxyethylene sorbitan fatty acid ester is from about 1:0.75 to about 1:20 w/w;
wherein the concentration of melphalan is from about 50 mg/ml to about 150 mg/ml.

12. The composition of claim 11, wherein the ratio of melphalan to polyoxyethylene sorbitan fatty acid ester is from about 1:1 to about 1:10 w/w.

13. The composition of claim 11, wherein the concentration of melphalan is from about 70 mg/ml to about 120 mg/ml.

14. The composition of claim 11, wherein the polyoxyethylene sorbitan fatty acid ester is at least one of polysorbate 20, polysorbate 40, polysorbate 60, and polysorbate 80.

15. The composition of claim 11, wherein the polyoxyethylene sorbitan fatty acid ester is polysorbate 80.

16. The composition of claim 11 further comprising at least one of propylene glycol and polyethylene glycol.

17. The composition of claim 16, wherein the at least one of propylene glycol and polyethylene glycol propylene glycol is about 10% to about 90% by weight of the composition.

18. The composition of claim 11 further comprising ethanol, wherein ethanol is about 5% to about 50% by weight of the composition.

19. The composition of claim 11 further comprising an antioxidant, wherein the antioxidant is at least one of tocopherol or its derivatives, thioglycerol, and monothioglycerol.

20. The composition of claim 11, wherein when the composition is diluted for administration to a, melphalan concentration of between about 0.1 mg/ml to about 3 mg/ml, the diluted composition is physically stable up to about 4 hours.

21. An aqueous injectable solution of melphalan produced by diluting a concentrated non-aqueous pharmaceutical composition comprising:
melphalan or a pharmaceutically acceptable salt thereof; and
a polyoxyethylene sorbitan fatty acid ester;
wherein the ratio of melphalan to polyoxyethylene sorbitan fatty acid ester is from about 1:0.75 to about 1:20 w/w;
wherein the concentration of melphalan in the concentrated composition is from about 50 mg/ml to about 150 mg/ml;
wherein the concentration of melphalan in the aqueous solution is from about about 0.1 mg/ml to about 3 mg/ml; and
wherein the aqueous solution is stable up to about 4 hours.

22. The solution of claim 21, wherein the ratio of melphalan to polyoxyethylene sorbitan fatty acid ester is from about 1:1 to about 1:10 w/w.

23. The solution of claim 21, wherein the polyoxyethylene sorbitan fatty acid ester is at least one of polysorbate 20, polysorbate 40, polysorbate 60, and polysorbate 80.

24. The solution of claim 21, wherein the polyoxyethylene sorbitan fatty acid ester is polysorbate 80.

25. The solution of claim 21 further comprising at least one of propylene glycol and polyethylene glycol.

26. The solution of claim 21 further comprising ethanol.

27. The solution of claim 21 further comprising an antioxidant, wherein the antioxidant is at least one of tocopherol or its derivatives, thioglycerol, and monothioglycerol.

28. A diluted injectable stable aqueous solution of melphalan comprising:
melphalan or a pharmaceutically acceptable salt thereof; and
a polyoxyethylene sorbitan fatty acid ester;
wherein the ratio of melphalan to polyoxyethylene sorbitan fatty acid ester is from about 1:0.75 to about 1:20 w/w;
wherein the solution is stable up to about 4 hours when the concentration of melphalan is from about 0.1 mg/ml to about 3 mg/ml.

29. A method of treating a patient in need of melphalan comprising parenterally administering to said patient an aqueous solution of melphalan comprising:
melphalan or a pharmaceutically acceptable salt thereof at a concentration of from about 0.1 mg/ml to about 3 mg/ml; and
a polyoxyethylene sorbitan fatty acid ester;
wherein the ratio of melphalan to polyoxyethylene sorbitan fatty acid ester is from about 1:0.75 to about 1:20 w/w;
wherein the solution is stable up to about 4 hours.

30. The method of claim 29, wherein the ratio of melphalan to polyoxyethylene sorbitan fatty acid ester in the solution is from about 1:1 to about 1:10 w/w.

31. The method of claim 29, wherein the polyoxyethylene sorbitan fatty acid ester is at least one of polysorbate 20, polysorbate 40, polysorbate 60, and polysorbate 80.

32. The method of claim 29, wherein the polyoxyethylene sorbitan fatty acid ester is polysorbate 80.

33. The method of claim 29 wherein the solution further comprises at least one of propylene glycol and polyethylene glycol.

34. The method of claim 29 wherein the solution further comprises ethanol.

35. The method of claim 29 wherein the solution further comprises an antioxidant, wherein the antioxidant is at least one of tocopherol or its derivatives, thioglycerol, and monothioglycerol.
